(19) United States
(12) Patent Application Publication
(10) Pub. No.: US 2008/0097410 A1
(43) Pub. Date: Apr. 24, 2008

(54) MEDICAL FLUID ASPIRATOR

(75) Inventor: Yuan-Yung Lee, Taipei City (TW)

Correspondence Address: PACIFIC HOSPITAL SUPPLY CO., LTD., P.O. BOX 108-00403, TAIPEI 106, omitted

(73) Assignee: PACIFIC HOSPITAL SUPPLY CO., LTD., Taipei City (TW)

(21) Appl. No.: 11/428,567

(22) Filed: Jul. 4, 2006

(51) Int. Cl. A61M 27/00

(52) U.S. Cl. 604/540

(57) ABSTRACT

A medical fluid aspirator, which includes a barrel attached with a suction tip, a tapered isolation sleeve detachably fastened to an inside connection tube inside the barrel and sealed with a gasket ring, an inner filter tube suspending in the isolation sleeve and tightly fastened to the rear side of the isolation sleeve for removing waste solid matters from waste fluid passing from the suction tip through the inner filter tube during the surgery, and an adapter that connect a suction tube to the rear side of the inner filter tube.

### BACKGROUND OF THE INVENTION

1. Field of the Invention

The present invention relates to medical implements and more particularly, to a medical fluid aspirator, which uses an isolation sleeve and an inner filter tube to collect waste solid matters when drawing waste fluid from the incision during a surgery.

2. Description of the Related Art

The body of a human being is composed of skeleton, tendons, blood vessels, muscles and blood. A human being eats food, drinks water, and breaths air to keep the life. However, bacteria and virus or an accident may cause a human being to suffer from a sick or injury. When a human being may have to receive a surgery in order to cure a serious sick or injury. During a surgical incision, waste fluid, muscle and/or bone chips, hairs and other waste matters must be quickly removed from the incision so that the surgery can be performed smoothly. A vacuum pump with a suction implement is usually used to remove waste fluid and waste solid matters from the incision during a surgery. FIGS. 8 and 9 show a conventional medical fluid aspirator for this purpose. According to this design, the medical fluid aspirator comprises a barrel A, which has a small front hole A1 and a big rear hole A2 in communication with the small front hole A1, a suction tube B connected to the small front hole A1, an inner tube C, which is mounted inside the barrel A and which has a plurality of through holes Cl cut through the peripheral wall, a front open end C2, and a rear close end C3, a holder block C4, which is fastened to the rear close end C3 of the inner tube C and has a plurality of through holes C41, and a rear cap D, which is capped on the holder block C4. This design of medical fluid aspirator is still not satisfactory in function because of the following drawbacks:

1. After installation of the inner tube C in the barrel A, the front open end C2 is coupled to an inside extension tube of the barrel A. During the operation of the vacuum pump to draw waste fluid and solid waste matters from the incision, waste fluid may flow through a gap between the front open end C2 of the inner tube C and the inside extension tube of the barrel A. In this case, waste solid matters may be carried with waste fluid through the gap between the front open end C2 of the inner tube C and the inside extension tube of the barrel A to further block the barrel A.

2. The inner tube C is fastened to the inside extension tube of the barrel A. When removing the inner tube C from the barrel A after the surgery, waste fluid and waste solid matters may be scattered all over the floor.

3. After installation of the inner tube C in the barrel A, the front open end C2 of the inner tube C is coupled to the inside extension tube of the barrel A. Because this design of medical fluid aspirator does not use any fastening means to affix the front open end C2 of the inner tube C to the inside extension tube of the barrel A, the inner tube C may be vibrated and biased during the suction action, thereby affecting the performance of the surgery.

### SUMMARY OF THE INVENTION

The present invention has been accomplished under the circumstances in view. It is the main object of the present invention to provide to a medical fluid aspirator, which prevents a leakage during the operation. It is another object of the present invention to provide a medical fluid aspirator, which removes waste solid matters from waste fluid passing through, and keeps the collected waste solid matters in place, preventing scattering of the collected waste solid matters after the surgery.

To achieve these and other objects of the present invention, the medical fluid aspirator comprises a barrel, the barrel having a front suction hole, an inside receiving chamber, a rear connection portion extending around one side of the barrel remote from the front suction hole, an inside connection tube suspending in the inside receiving chamber in communication with the front suction hole; a gasket ring fastened to the periphery of the inside connection tube within the inside receiving chamber; an isolation sleeve inserted into the inside receiving chamber of the barrel, the isolation sleeve having a front inlet sleeved onto the inside connection tube of the barrel and tightly stopped against the gasket ring, a tapered accommodation chamber in communication with the front inlet, and a rear locating flange extending around a rear side of the isolation sleeve remote from the front inlet; an inner filter tube inserted into the tapered accommodation chamber of the isolation sleeve, the inner filter tube having a base stopped outside the rear locating flange of the isolation sleeve, a locating portion forced into engagement with the rear locating flange of the isolation sleeve, a close-end tube extending from the locating portion and suspending in the tapered accommodation chamber of the isolation sleeve, an axial fluid passage cut through the center of the base, and a plurality of filter holes cut through the periphery of the closed-end tube in communication between the axial fluid passage and the tapered accommodation chamber of the isolation sleeve; and an adapter fastened to the rear connection portion of the barrel and stopped against the base of the inner filter tube, the adapter having a drainage hole adapted to guide fluid out of the axial fluid passage of the inner filter tube. After the surgery, the filtered waste solid matters are kept in the tapered accommodation chamber inside the isolation sleeve around the close-end tube of the inner filter tube, and therefore the filtered waste solid matters can be properly disposed off.

### BRIEF DESCRIPTION OF THE DRAWINGS

FIG. 1 is a perspective view of a medical fluid aspirator in accordance with the present invention.

FIG. 2 is an exploded view of the medical fluid aspirator in accordance with the present invention.

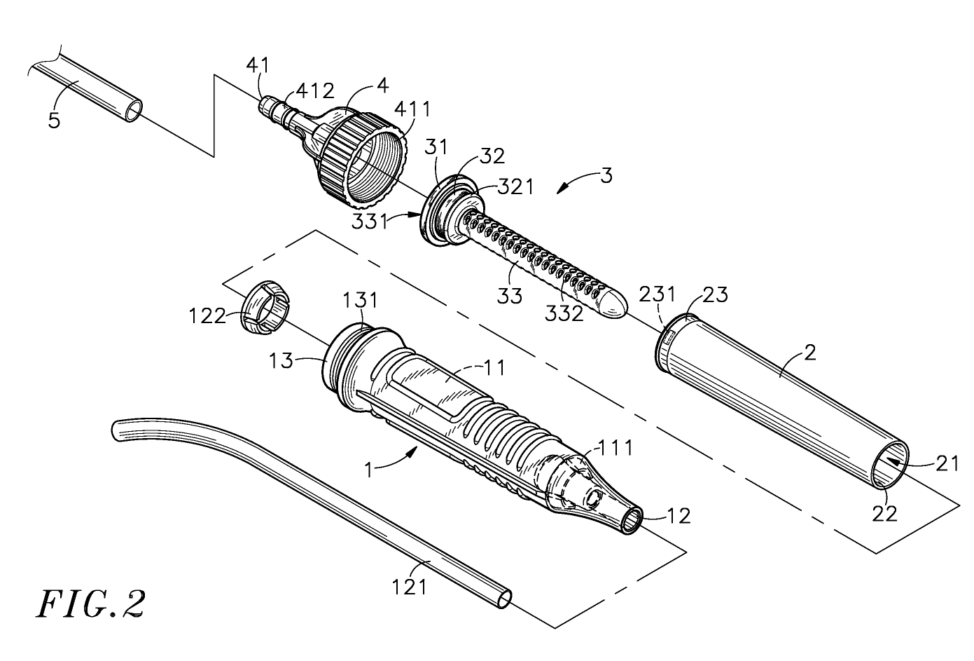

FIG. 3 is a perspective sectional side view of the medial fluid aspirator in accordance with the present invention.

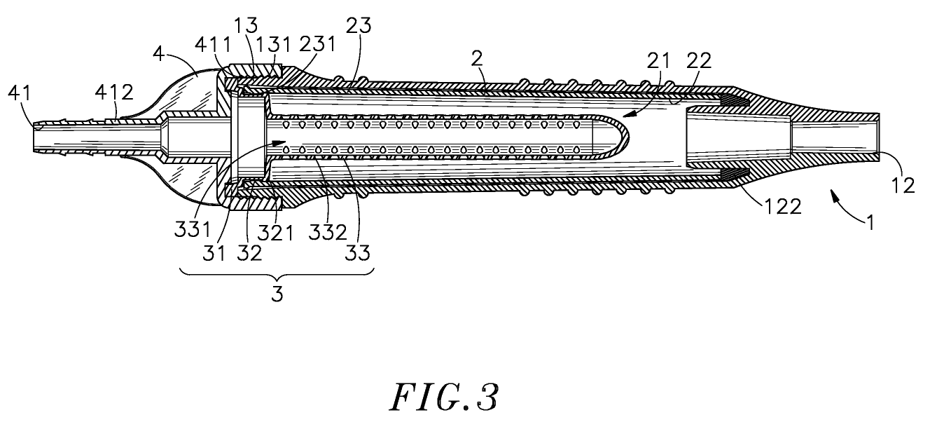

FIG. 4 is a schematic drawing showing an application example of the present invention.

FIG. 5 is a sectional side view of the medical fluid aspirator in accordance with the present invention.

FIG. 6 is a schematic sectional view of the present invention, showing removal of the isolation sleeve with the inner filter tube from the barrel.

FIG. 7 is a schematic sectional view of the present invention, showing the inner filter tube separated from the isolation sleeve.

FIG. 8 is a sectional side view of a medical fluid aspirator according to the prior art.

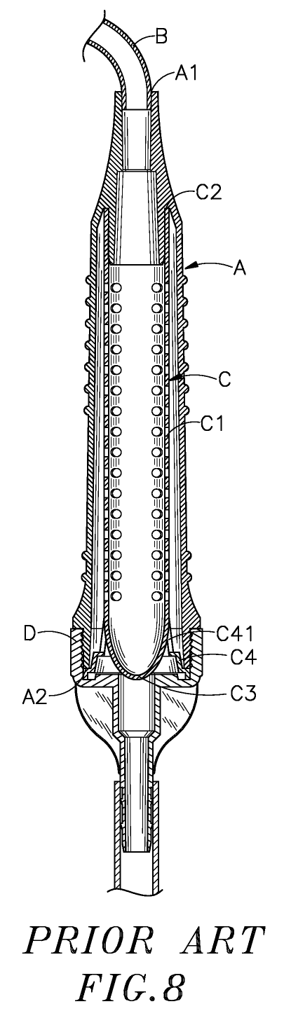

FIG. 9 is a sectional exploded view of the medical fluid aspirator according to the prior art.

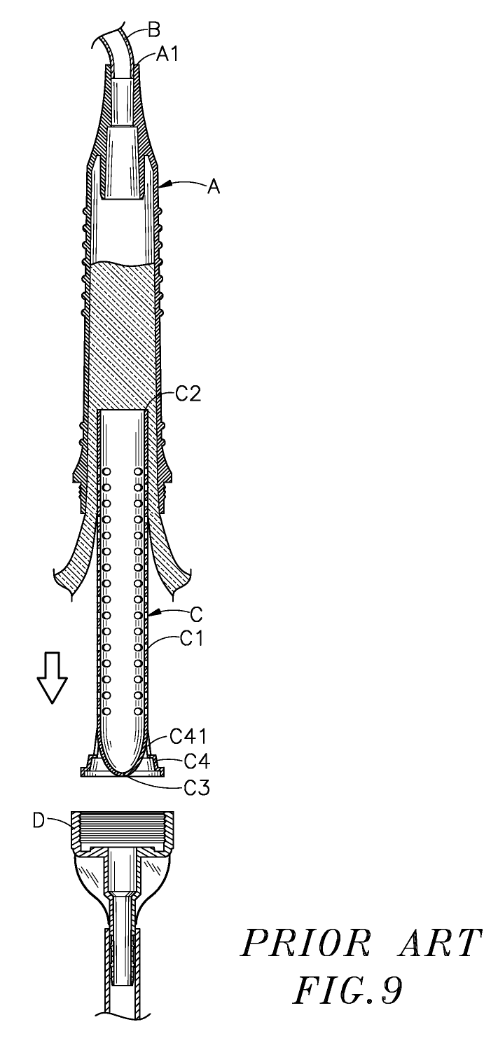

### DETAILED DESCRIPTION OF THE PREFERRED EMBODIMENT

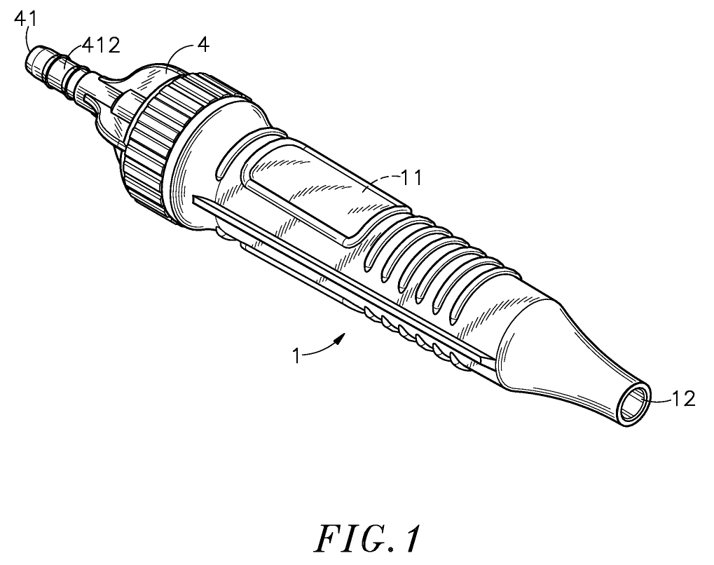

Referring to FIGS. 1˜3, a medical fluid aspirator in accordance with the present invention is shown comprised of a barrel 1, an isolation sleeve 2, an inner filter tube 3, and an adapter 4.

The barrel 1 has an inside receiving chamber 11, a front suction hole 12 disposed at one side, namely, the front side and attached with a suction tip 121, an inside connection tube 111 fixedly mounted on the inside in communication between the inside receiving chamber 11 and the front suction hole 12, a gasket ring 122 fastened to the periphery of the inside connection tube 111 to seal a gap, a rear connection portion 13 disposed at the other side, namely, the rear side, an outer thread 131 extending around the periphery of the rear connection portion 13.

The isolation sleeve 2 is a tapered sleeve, having a tapered accommodation chamber 21, an inlet 22 at the narrow front side of the tapered accommodation chamber 21, a rear locating flange 23 around the broad rear side of the tapered accommodation chamber 21, and at least one retaining block 231 on the inside wall of the rear locating flange 23.

The inner filter tube 3 has a base 31 at one side, namely, the rear side, a close-end tube 33 at the other side, namely, the front side, a locating portion 32 connected between the base 31 and the close-end tube 33, a retaining flange 321 formed integral with one end of the locating portion 32 adjacent to the close-end tube 33 and extending around the periphery of the locating portion 32, an axial fluid passage 331 axially extending through the center of the base 31 and the locating portion 32 into the inside of the close-end tube 33, and a plurality of filter holes 332 cut through the periphery of the close-end tube 33 in communication with the axial fluid passage 331.

The adapter 4 has a front inner thread 411, a rear connection portion 412, and a drainage hole 41 axially extending through the two distal ends. The rear connection portion 412 of the adapter 4 is connected to a suction tube 5.

During assembly process, the isolation sleeve 2 is inserted into the inside receiving chamber 11 of the barrel 1 to connect the inlet 22 to the inside connection tube 111 and to stop the periphery of the inlet 22 (i.e., the front end of the isolation sleeve 2) against the gasket ring 122, and then the close-end tube 33 of the inner filter tube 3 is inserted into the tapered accommodation chamber 21 of the isolation sleeve 2 to force the locating portion 32 and the retaining flange 321 into engagement with the rear locating flange 23 and the retaining block 231 respectively, and then the front inner thread 411 of the adapter 4 is threaded onto the outer thread 131 of the barrel 1, and then the rear connection portion 412 of the adapter 4 is connected to the suction tube 5. Thus, the medical fluid aspirator is assembled for application.

In an alternate form of the present invention, the isolation sleeve 2 and the inner filter tube 3 are made in integrity, keeping the closed-end tube 33 suspending in the tapered accommodation chamber 21 of the isolation sleeve 2.

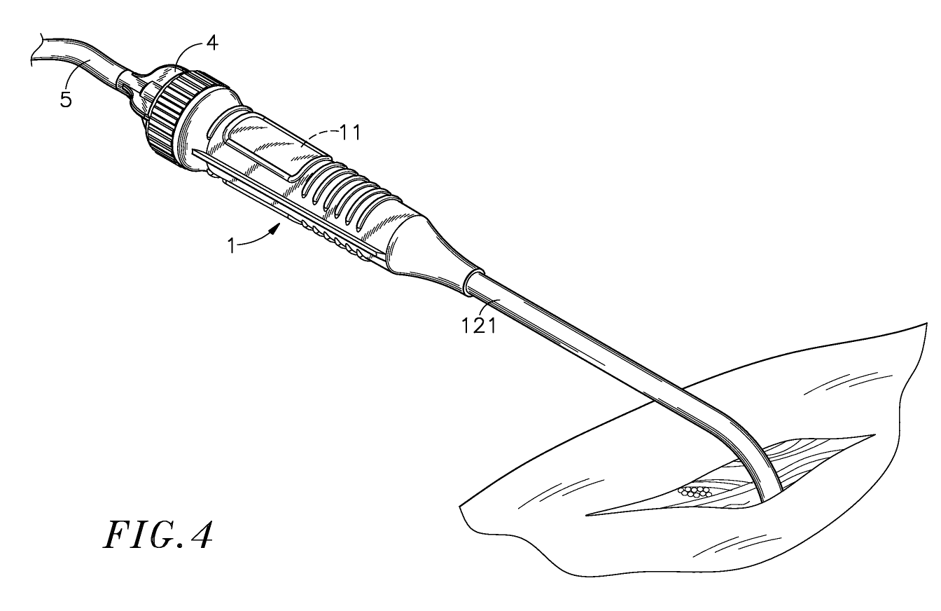

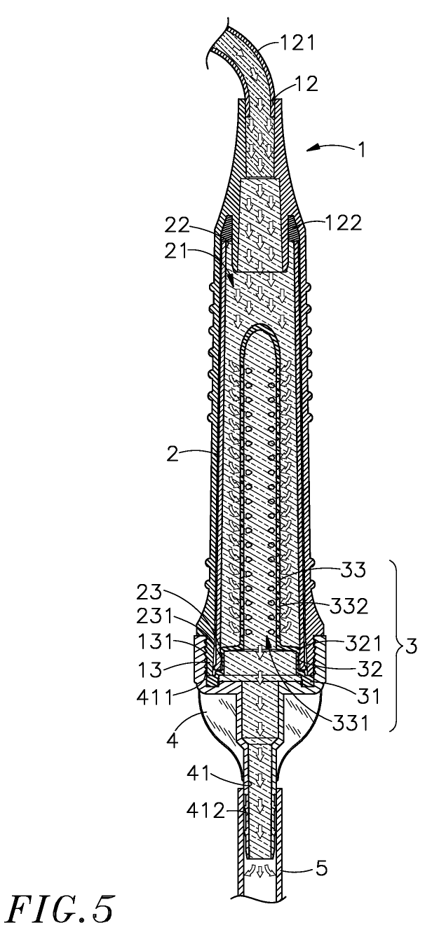

Referring to FIGS. 4 and 5, when in use, the suction tip 121 of the barrel 1 is approached to an incision so that the suction pump (not shown) that is connected to the suction tube 5 draw waste fluid and waste solid matters from the incision into the tapered accommodation chamber 21 of the isolation sleeve 2 through the front suction hole 12 and the inside connection tube 111 of the barrel 1 and then from the tapered accommodation chamber 21 into the suction tube 5 through the filter holes 332 and axial fluid passage 331 of the inner filter tube 3 and the drainage hole 41 of the adapter 4. At this time, the filter holes 332 remove solid matters of diameters greater than the filter holes 332 from the waste fluid, preventing blocking of the suction tube 5 and the suction pump by waste solid matters.

As stated above, the front end of the isolation sleeve 2 is stopped against the gasket ring 122 so that waste fluid does not leak out of the isolation sleeve 2.

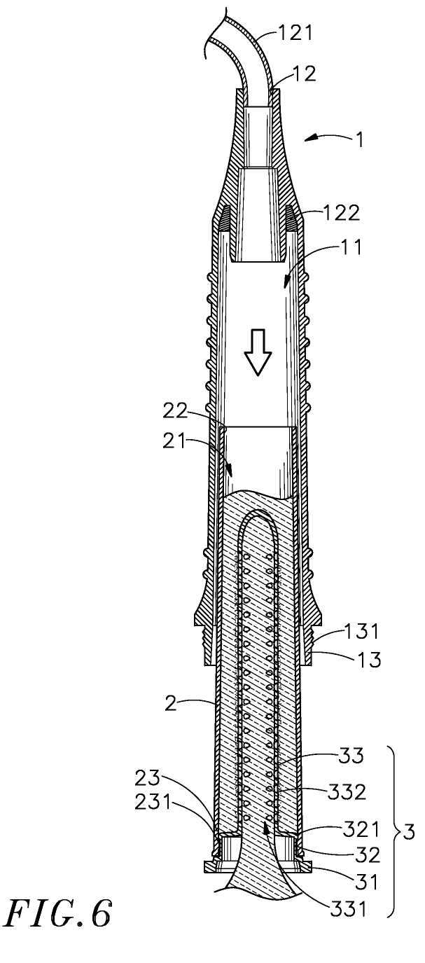

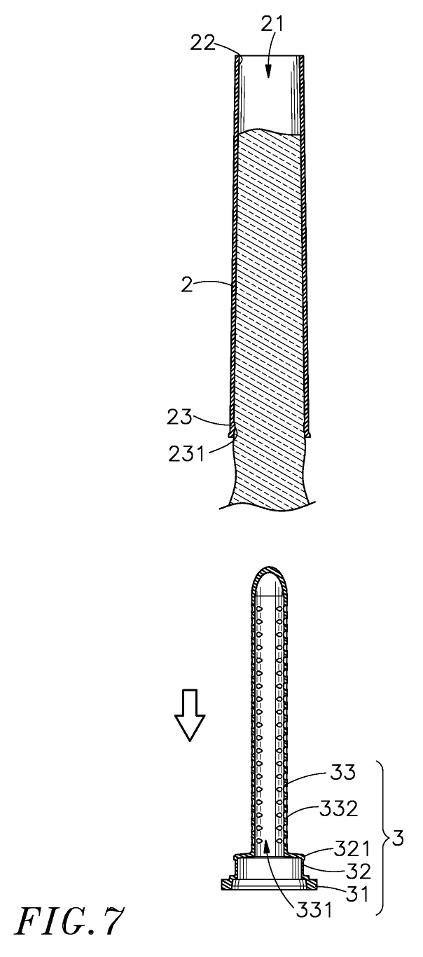

Referring to FIGS. 6 and 7, after the use of the medical fluid aspirator, the isolation sleeve 2 and the inner filter tube 3 must be removed from the barrel 1 for cleaning and sterilization. At this time, disconnect the front inner thread 411 of the adapter 4 from the outer thread 131 of the barrel 1, and then remove the isolation sleeve 2 and the inner filter tube 3 from the inside receiving chamber 11 of the barrel 1. After removal of the isolation sleeve 2 and the inner filter tube 3 from the barrel 1, the residual waste solid matters are kept in the tapered accommodation chamber 21 of the isolation sleeve 2 around the close-end tube 33 of the inner filter tube 3 and can be well collected and properly disposed off. After disconnection of the locating portion 32 of the inner filter tube 3 from the rear locating flange 23 of the isolation sleeve 2, the barrel 1, the isolation sleeve 2, the inner filter tube 3 and the adapter 4 are well washed and sterilized for a repeated use.

As stated above, the inside receiving chamber 11 of the barrel 1 receives the isolation sleeve 2 and the inner filter tube 3, and the isolation sleeve 2 is fastened to the inside connection tube 111 of the barrel 1 and firmly stopped against the gasket ring 122, preventing a leakage.

In actual practice, the medical fluid aspirator of the present invention has the follow benefits:

1. The inlet 22 of the isolation sleeve 2 is connected to the inside connection tube 111 of the barrel 1, and the gasket ring 122 seals the gap between the isolation sleeve 2 and the inside connection tube 111, preventing a leakage.

2. By means of the suction tip 121, waste fluid and waste solid matters can be efficiently drawn through the medical fluid aspirator into the suction tube 5. Further, the filter holes 332 of the close-end tube 33 of the inner filter tube 3 remove waste solid matters from waste fluid, preventing block of the suction tube 5 and the suction pump by waste solid matters. After removal of the isolation sleeve 2 with the inner filter tube 3 from the barrel 1, waste solid matters are kept in the tapered accommodation chamber 21 of the isolation sleeve 2 around the close-end tube 33 of the inner filter tube 3 and can be properly disposed off.

3. The inlet 22 of the isolation sleeve 2 is fastened to the inside connection tube 111 of the barrel 1 and firmly stopped against the gasket ring 122. Therefore, the isolation sleeve 2 does not vibrate relative to the barrel 1 during the operation of the medical fluid aspirator.

Although a particular embodiment of the invention has been described in detail for purposes of illustration, various modifications and enhancements may be made without departing from the spirit and scope of the invention.

What the invention claimed is:

1. A medical fluid aspirator comprising:
a barrel, said barrel having a front suction hole, an inside receiving chamber, a rear connection portion extending around one side of said barrel remote from said front suction hole, an inside connection tube suspending in said inside receiving chamber in communication with said front suction hole;
a gasket ring fastened to the periphery of said inside connection tube within said inside receiving chamber;
an isolation sleeve inserted into said inside receiving chamber of said barrel, said isolation sleeve having a front inlet sleeved onto said inside connection tube of said barrel and tightly stopped against said gasket ring, a tapered accommodation chamber in communication with said front inlet, and a rear locating flange extending around a rear side of said isolation sleeve remote from said front inlet;
an inner filter tube inserted into said tapered accommodation chamber of said isolation sleeve, said inner filter tube having a base stopped outside said rear locating flange of said isolation sleeve, a locating portion forced into engagement with said rear locating flange of said isolation sleeve, a filter tube extending from said locating portion and suspending in said tapered accommodation chamber of said isolation sleeve, an axial passage cut through the center of said base, and a plurality of filter holes cut through the periphery of said filter tube in communication between said axial fluid passage and said tapered accommodation chamber of said isolation sleeve; and
an adapter fastened to said rear connection portion of said barrel and stopped against said base of said inner filter tube, said adapter having a drainage hole adapted to guide fluid out of said axial fluid passage of said inner filter tube.

2. The medical fluid aspirator as claimed in claim 1, further comprising a suction tip fastened to the front suction hole of said barrel.

3. The medical fluid aspirator as claimed in claim 1, wherein said rear connection portion of said barrel has an outer thread for the mounting of said adapter.

4. The medical fluid aspirator as claimed in claim 1, wherein said isolation sleeve is a tapered sleeve, having at least one retaining block inside said rear locating flange for fastening to said locating portion of said inner filter tube.

5. The medical fluid aspirator as claimed in claim 1, wherein said filter tube is a close-end tube.

6. The medical fluid aspirator as claimed in claim 1, wherein said inner filter tube has a retaining flange extending around the periphery of one end of said locating portion.

7. The medical fluid aspirator as claimed in claim 1, wherein said adapter has a front inner thread disposed at a front side thereof for fastening to said rear connection portion of said barrel, and a rear connection portion disposed at a rear side thereof around said drainage hole for the connection of a suction tube.

8. The medical fluid aspirator as claimed in claim 1, wherein said isolation sleeve is formed integral with said inner filter tube.
